SEQUENCE LISTING (18 sequences)

SEQ ID NO: 1 - DNA, 27 bp, Artificial Sequence
  PCR primer SaAEN
ccgaattccatggtgaggggaaaaaca

SEQ ID NO: 2 - DNA, 27 bp, Artificial Sequence
  PCR primer AtAEN
ccgaattccatggtgaggggcaaaact

SEQ ID NO: 3 - DNA, 32 bp, Artificial Sequence
  PCR primer SaAEB
ccgaattcggatcctcactttctggaagaaca

SEQ ID NO: 4 - DNA, 32 bp, Artificial Sequence
  PCR primer AtAEB
ccgaattcggatcctcactttcttgaagaaca

SEQ ID NO: 5 - DNA, 27 bp, Artificial Sequence
  PCR primer SaBEN
ccgaattccatgggaaggggtagggtt

SEQ ID NO: 6 - DNA, 27 bp, Artificial Sequence
  PCR primer AtBEN
ccgaattccatgggaagaggtagggtt

SEQ ID NO: 7 - DNA, 32 bp, Artificial Sequence
  PCR primer SaBEB
ccgaattcggatccctactcgttcgaagtggt

SEQ ID NO: 8 - DNA, 32 bp, Artificial Sequence
  PCR primer AtBEB
ccgaattcggatccctactcgttcgtagtggt

SEQ ID NO: 9 - DNA, 27 bp, Artificial Sequence
  PCR primer NtFPF-EN
caggaattccatggctggagtttgggt

SEQ ID NO: 10 - DNA, 33 bp, Artificial Sequence
  PCR primer NtFPF-EB
caggaattcggatccttatcatatgtctctaac

SEQ ID NO: 11 - DNA, 665 bp, Arabidopsis thaliana
tcaagaaaccaaaactaaggagaggaagaagaagatcatgtcaggcgtgtgggtcttcaagaacggagtgataagacttgtggagaaccctaaccagtcaggatccgacacacagaaccgaaggaaagtgatggtctatttaccgacaggagaagtggtctcatcttactcgacgctcgagcagatcctccagagtcttggatgggagagatactttggcggcggcgacacagatctcctccaattccacaaacgctcctccattgatctcatttccctgcctagagatttcaccaaattcaactccgtttacatgtacgatatcgtcgtcaagaaccctaattacttccatgtccgagactcccattgattcttcttttgtgtgttggtgtgtgtgttgtgttcggttgttttataatttagagtttgttgggttttgttttatagttgcatgtgcatgttttatttgttttggacgttggaagttgtgttacgtaattctggtttgatgaaagttgagattaagaagggacatgaattagggagaactcaatttcaaaattaattgttctcttccttagcatgattgttctcttcactggttgtaattgaaagatattctctttggctcagtggatccaaattgttttctatcttctatttaa

SEQ ID NO: 12 - DNA, 610 bp, Sinapis alba
aacacactaaaccatcgcatactctctctctttctctcatctctctcaagaagatcatgtcaggcgtgtgggtgttcaagaacggagtgataaggcttgtggagaaccctaaccagtcaggaggcgacacaaatagccgaaggaaagtgatggtctatttaccgacaggagaagtgatctcatcttactccacgctcgagcagatcctccggagtcttggatgggagaggtacttcggtggcggcgacacagatcttctccaattccacaaacgctcatccattgatctcatctctctccctaaagatttcaccaaattcagctctgtttacatgtacgatattgtcgtcaagaaccctaattacttccacgtccgagactccaattaaacccttttcttttttttatgtttgtgtgtgttgttttgttgtttttaattgaatgttctgttgggttttgttttagttgcattgcatgtgcatgttttgtttgttgttgacgttgagagttgtgtaactgtgtatgtaattgtgctttaatgaaagttgagattaataagaagtttggtttagggagaaaaaaaaaaaaaaaaaaaaaaaaaaaaaaaaaa

SEQ ID NO: 13 - DNA, 1000 bp, Arabidopsis thaliana
cctatacatacacaaaccctttatcctcgaaagcttcctcctggttaggtttttatcaaacccttttagccaatcgggaaattaactaaagaagaagatatggtgaggggcaaaactcagatgaagagaatagagaatgcaacaagcagacaagtgactttctccaaaagaaggaatggtttgttgaagaaagcctttgagctctcagtgctttgtgatgctgaagtttctcttatcatcttctctcctaaaggcaaactttatgaattcgccagctccaatatgcaagataccatagatcgttatctgaggcatactaaggatcgagtcagcaccaaaccggtttctgaagaaaatatgcagcatttgaaatatgaagcagcaaacatgatgaagaaaattgaacaactcgaagcttctaaacgtaaactcttgggagaaggcataggaacatgctcaatcgaggagctgcaacagattgagcaacagcttgagaaaagtgtcaaatgtattcgagcaagaaagactcaagtgtttaaggaacaaattgagcagctcaagcaaaaggagaaagctctagctgcagaaaacgagaagctctctgaaaagtggggatctcatgaaagcgaagtttggtcaaataagaatcaagaaagtactggaagaggtgatgaagagagtagcccaagttctgaagtagagacgcaattgttcattgggttaccttgttcttcaagaaagtgatctccactcaacaaaaaaaacaaaaaaacaaactaagtaaaaacaggtttagcagtactgagagtgtataaggacaagtaacaaaactctaaagctgcttctctcttgttgtcttatttctctctcactctctctctctcagtcacttctttaatgattctgaactgcttgtgttatgtatctatgttcttacacttgcctctctagaatgtaattttgattatgttttaaaaaaaaaaaaaaaaaaaaaaaaaaa

SEQ ID NO: 14 - DNA, 882 bp, Sinapis alba
ggatattaaacaaggagaaattaagagatggtgaggggaaaaacacagatgaagcgaatagagaatgcaacaagcagacaagtcactttctctaaacgaaggaatggtttgttgaagaaagcctttgagctctcagtgctttgtgatgctgaagtttctcttatcatcttctctcctaagggaaaactttatgagttcgccagctccaatatgcaagataccgtagatcgttatctgaggcacaccaaggatcgagtcagcagcaaaccggtttctgaagaaaatatgcagcatttcaaacatgaagcagcaaacatgatgaagaaaattgaacaacttgaagcttccaaacgtaaactcttgggagaaggcataggatcatgctcgattgaggagctgcagcaaattgagcaacaacttgagaaaagtgtcaaatgtgttcgagcaagaaagactcaagtgtttaaggaacaaattgagcagctcaagcagaaggagaaagctctagctgcagaaaacgagaagctcgctgaaaagtggggatctcatgaaatcgaagtttggtcgaataagaaacaagaaagtggacgaggtgacgaggagagtagcccaagttctgaagtagagacacaattgttcattgggttaccttgttcttccagaaagtgaagcaactactactctaagaacaacaaaaacaaaataaaacaaagcaaaaaaaactatatgtaaaaacgggttagcagtgagtgtataaggacaagtaacaaaactctaaagctgcttctcttgttgtcttctttttctctctctcactctcttcttcaaaactgaactgcctgtggtatgtatctatgttcttatacacttgccctctctaga

SEQ ID NO: 15 - DNA, 1058 bp, Arabidopsis thaliana
cccagagagacataagaaagaaagagagagagagatactttggtcatttcagggttgtcgtttctctctcttgttcttgagattttgaagagagagagatatgggaagaggtagggttcagctgaagaggatagagaacaagatcaataggcaagttactttctcaaagagaaggtctggtttgctcaagaaagctcatgagatctctgttctctgcgatgctgaggttgctctcatcgtcttctcttccaaaggcaaactcttcgaatattccaccgactcttgcatggagaggatacttgaacgctatgatcgctatttatattcagacaaacaacttgttggccgagacgtttcacaaagtgaaaattgggttctagaacatgctaagctcaaggcaagagttgaggtacttgagaagaacaaaaggaattttatgggggaagatcttgattcgttgagcttgaaggagctccaaagcttggagcatcagctcgatgcagctatcaagagcattaggtcaagaaagaaccaagctatgttcgaatccatatctgcgctccagaagaaggataaagccttgcaagatcacaacaattcgcttctcaaaaagattaaggagagggagaagaaaacgggtcagcaagaaggacaattagtccaatgctccaactcttcttcagttcttctgcctcaatactgcgtaacctcctccagagatggctttgtggagagagttgggggagagaacggtggtgcatcgtcgttgacggaaccaaactctctgcttccggcttggatgttacgtcctaccactacgaacgagtagaactatctcactctttataatataatgataatataattaatgtttaatattttcataacattcagcatttttttggtgacttatactcattattaataccgatatgttttagctagtcatattatatgtatgatggaactccgttgtcgagacgtatgtacgtaagctatcattagattcactgcgtcttaagaacaaagattcatatcttggtaatgatttctcatga

SEQ ID NO: 16 - DNA, 946 bp, Sinapis alba
aaagaaatagagagagagaggtcctttggacatttcaaggttatcgtttctctctcttgttcttgagattttgaggagagagatgggaaggggtagggttcagctgaagaggatagaaaacaagatcaataggcaagttactttctcaaagagaaggtctggtttgctcaagaaagctcatgagatctctgttctctgcgatgctgaggttgctctcgtcatcttctcttccaaaggcaaactcttcgaatattccaccgactcttgcatggaaaagatacttgaacgctatgatcgctatttgtattcagacaaacaacttgtaggccgagacatttcacagagtgaaaattgggttctagaacatgctaagctcaaggcaagagttgaggtactcgagaagaacaaaaggaattttatgggggaagatcttgattcgttgagcttaaaggagctccaaagcttggagcaccagctccatgctgctatcaagagcattaggtcaagaaagaaccaagctatgttcgaatccatatcagcgctccagaagaaggataaggtcttgcaagatcacaacaatgcgcttctcaaaaagattaaggagagggagaagaacacggttcatcaagaagtacaactaatccaatgctccaacaattcttcaattctccagccccaatactgcttaacctcctccagagatggctttgtgggtagagttgagggagagaacgaaggtgcatcgtcgttggcggaaccaaactctctccttccagcttggatgttacgccctacaacgaacgaatgaatagaattatctctatataatacgatacattaacgtttaatattctcatagcattttggtgtatttactgaactgatataagttgactttttaactagttatataaaattttatatataatgatggaattacaatgac

SEQ ID NO: 17 - DNA, 540 bp, Arabidopsis thaliana
aagcttctaaacgtaaactcttgggagaaggcataggaacatgctcaatcgaggagctgcaacagattgagcaacagcttgagaaaagtgtcaaatgtattcgagcaagaaagactcaagtgtttaaggaacaaattgagcagctcaagcaaaaggagaaagctctagctgcagaaaacgagaagctctctgaaaagtggggatctcatgaaagcgaagtttggtcaaataagaatcaagaaagtactggaagaggtgatgaagagagtagcccaagttctgaagtagagacgcaattgttcattgggttaccttgttcttcaagaaagtgatctccactcaacaaaaaaaacaaaaaaacaaactaagtaaaaacaggtttagcagtactgagagtgtataaggacaagtaacaaaactctaaagctgcttctctcttgttgtcttatttctctctcactctctctctctcagtcacttctttaatgattctgaactgcttgtgttatgtatctatgttcttacacttgcctctctaga

SEQ ID NO: 18 - DNA, 540 bp, Arabidopsis thaliana
aagcttggagcatcagctcgatgcagctatcaagagcattaggtcaagaaagaaccaagctatgttcgaatccatatctgcgctccagaagaaggataaagccttgcaagatcacaacaattcgcttctcaaaaagattaaggagagggagaagaaaacgggtcagcaagaaggacaattagtccaatgctccaactcttcttcagttcttctgcctcaatactgcgtaacctcctccagagatggctttgtggagagagttgggggagagaacggtggtgcatcgtcgttgacggaaccaaactctctgcttccggcttggatgttacgtcctaccactacgaacgagtagaactatctcactctttataatataatgataatataattaatgtttaatattttcataacattcagcatttttttggtgacttatactcattattaataccgatatgttttagctagtcatattatatgtatgatggaactccgttgtcgagagaattcctgcagcccgggggatccactagttctaga

(19) United States
(12) Patent Application Publication
(10) Pub. No.: US 2006/0206965 A1
(43) Pub. Date: Sep. 14, 2006

(54) Combination of genes for the regulation of the induction of flowering in useful and ornamental plants

(76) Inventors: Roland Gleissner, Zurich (CH); Siegbert Melzer, Elgg (CH); Birgit Kurz, Raterschen (CH); Klaus Apel, Radolfzell (DE)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(21) Appl. No.: 11/200,465

(22) Filed: Aug. 9, 2005

Related U.S. Application Data
(63) Continuation of application No. 09/646,345, filed on Oct. 30, 2000, now abandoned.

(30) Foreign Application Priority Data
Mar. 19, 1998 (CH) 657/98

(51) Int. Cl. A01H 1/00; C12N 15/82; C12N 15/87

(52) U.S. Cl. 800/287; 800/294

(57) ABSTRACT

The invention relates to the combination of genes MADSA, FPF1 or MADSB and FPF1 for flowering induction. As a result, the flowering time of useful and ornamental plants can be regulated while taking a balanced development of the plants into consideration.

This application claims the benefit of U.S. Nonprovisional application 09/646,345, which is a 371 of PCT/CH99/00122.

The present invention relates to the regulation of the time of flowering in useful and ornamental plants.

All the prior-art processes for the regulation of flowering are based on the overexpression of an individual gene. Since the induction of flowering, as described in the following, is regulated by a network of genes participating therein, of which many have still not been cloned and characterized, this path is not optimal for a graduated development of the plants.

The transition from vegetative growth to flowering is a clearly visible shift to a new development program for a plant. It shows a change in function of the apical meristem which passes from the formation of leaves to the formation of flowers. This morphogenetic alteration is either controlled by endogenous factors by which the genetic program for flowering is “engaged” after a certain period of vegetative growth, that is, after a definite number of leaves have been produced, or on the other hand by different environmental conditions. The most important and most extensively investigated environmental conditions are low temperatures (vernalization) and the length of daylight (photoperiod). In greenhouses these environmental conditions can be adapted in order to ensure an optimal growth of plants or in order to achieve as great a success in reproduction as possible during the transition from vegetative growth to flowering. This requires however a significant use of nonrenewable energy. Under field conditions this is not possible without additional measures. It is thus a goal of the classical cultivation of plants to select varieties with a definite time of flowering. In the case of early flowering varieties it would then be possible to cultivate important cultivated plants even in regions in which they do not normally reach complete maturity. The selection of early flowering varieties by classical cultivation is however very time-intensive.

Since it is known that the photoperiod is an important factor in the regulation of the time of flowering, stoppage and defoliation experiments have yielded indications that a previously unknown flowering stimulus is produced in the leaves of plants if they are exposed to a critical length of day. This signal is transported from the leaves via the phloem to the apical meristem. If this flowering stimulus has reached an apical meristem which is ready to react to this signal, flowering is initiated.

Flowering time mutants of Arabidopsis thaliana, which flower later or earlier than the corresponding wild-type of plants, play an important role in the clarification of the induction events in the leaves and the signal transmission path from the leaves to the meristem. In the case of Arabidopsis 13 different genes which play a role in the induction of flowering have been identified with the aid of late-flowering mutants. These genes can be classified by genetic investigations into three parallel signal transinduction paths (Koomneef, M. et al., Mol. Gen. Genet. 229, 57-66, 1991). The two genes cloned first which play a role in the determination of the time of flowering of Arabidopsis code regulatory proteins which are expressed constitutively. LUMINIDEPENDENS (LD) appears to influence the perception of light (Lee et al., Plant Cell 6, 75-83, 1994) and CONSTANS (CO) is necessary for the induction of flowering under daylight conditions (Putterill et al., Cell 80, 847-857, 1995). An additional gene which regulates the transition to flowering is FCA. This gene was cloned and it could be shown that the coded protein has two RNA binding sites and one protein interaction domain. It is thus assumed that FCA is involved in the regulation of the transcript maturation of genes which play a role in the induction of flowering (Macknight et al., Cell 89, 737-745, 1997).

In recent years noteworthy progress has been made with regard to the understanding of the regulation of the organogenesis of flowering. Genetic and molecular investigations of the development of flowering in Antirrhinum majus and Arabidopsis have led to the isolation of identity genes of the flowering meristem and the flowering organs which control flowering (Coen, E. S. and Meyerowitz, E. M., Nature 353, 31-37, 1991; Weigel, D. and Meyerowitz, E. M., Science 261, 1723-1726, 1993). All but two of these genes code gene products with an amino-terminal DNA-binding domain which have homologies to the DNA-binding domains of the transcription factors MCM1 of yeast and SRF of mammals. These domains were designated as MADS Box, an abbreviation of the names of the genes first cloned (Schwarz-Sommer et al., Science 250, 931-936, 1990). It could be demonstrated that the identity genes of the flowering organs are regulated in part by gene products of the identity genes of the flowering meristem such as FLORICAULA (FLO) in Antirrhinum (Hantke et al., Development 121, 27-35, 1995) and in Arabidopsis by the FLO homology LEAFY (LFY) and the MADS Box gene APETALA1 (AP1) (Weigel, D. and Meyerowitz, E. M., Science 261, 1723-1726, 1993). Moreover, it could be shown that TERMINAL FLOWER1 (TFL1), a gene which is responsible for the regulation of the formation of the flowering meristem and the maintenance of the inflorescence meristem, interacts with the LFY and AP1 (Gustafson-Brown, C. et al., Cell 76, 131-143, 1994; Shannon S. and Meeks-Wagner, D. R., Plant Cell 5, 639-655, 1993; Weigel, D. et al., Cell 69, 843-859, 1992) and that CO interacts with LFY (Putterill, J. et al., supra).

It was also demonstrated that the constitutive expression of LFY, AP1, and CO leads to an advanced flowering in Arabidopsis thaliana (Weigel, D. and Nilsson, O., Nature 377, 495-500, 1995; Mandel, M. A. and Yanofsky, M. F., Nature 377, 522-544, 1995; Simon, R. et al., Nature 324, 59-62, 1996). The ectopic expression of these genes under the control of the cauliflower mosaic virus (CaMV) 35S promoter leads however to pleiotropic effects which strongly affect the yield of seed. Thus all three named genes (LFY, AP1, and CO) lead to a disposition of terminal fused flowers which suppress any additional onset of flowering in the inflorescence of Arabidopsis. The formation of a closed inflorescence with a premature terminal flower otherwise only results in Arabidopsis after mutations in the gene TERMINAL FLOWER1 (TFL1) which normally is switched on after the induction of flowering just below the inflorescence meristem (Bradley et al., Science 275, 80-83, 1997). The ectopic expression of the regulatory genes which are involved in flowering seem to repress TFL1. In the case of the LFY overexpression there is furthermore the formation of flowers in the leaf axes of transgenic Arabidopsis plants which develop buds with a greatly reduced yield of seeds.

Additional genes which influence the time of flowering are OsMADS1 (Chung, Y. -Y. et al., Plant Molecular Biology 26, 657-665, 1994) which leads in the case of a constitutive expression in transgenic tobacco plants to dwarf growth and shortened inflorescence as well as SPL3 (Cardon, G. H. et al., Plant Journal 12, 367-377, 1997).

The genes MADSA (Gene Bank Accession No. U25696, SEQ ID NO. 14) and MADSB (Gene Bank Accession No. U25695, SEQ ID NO. 16) (Menzel, S. et al., Plant Journal 9, 399-408, 1996) and FPF1 for SaFPF1 (EMBL Accession No. Y11987, SEQ ID NO. 12) were originally isolated from mustard (Sinapis alba). The ATFPF1 gene (EMBL Accession No. Y11988) was also isolated from Arabidopsis. (Kania, T. et al., Plant Cell 9, 1327-1338, 1997)). It could be shown that these genes are induced before LFY and AP1 in the apical meri stem after the induction of flowering.

MADSA and MADSB were identified with the use of the MADS Box coding region of the flowering organ identity gene AGAMOUS (AG) (Menzel et al., supra). The two genes are expressed during the transitional phase from vegetative growth to flowering in the apical meristem of Sinapis alba and Arabidopsis thaliana. RNA blot analyses have confirmed that the number of transcripts of the two genes is drastically increased shortly before the induction of flowering and that both genes are expressed earlier than the MADS Box genes AP1 and AG. In situ hybridizations have shown that the expression of the genes on the apical meristem of the induced plants is restricted during the early phases of reproductive development. The expression of MADSA is first demonstrable in the center of the meristem. In this region the earliest changes of an activated meristem can be demonstrated by classical physiological processes. MADSA could thus have an important function during the transition from vegetative growth to flowering. The Arabidopsis gene homologous to MADSB was also described as AGL8 by Mandel and Yanofsky (Plant Cell, 9, 1763-1771, 1995) (Gene Bank Accession Number U33473) while a sequence homologous to MADSA as EST (expressed sequence tag) (Newmann et al., Plant Physiol. 106, 1241-1255, 1994) was isolated from Arabidopsis (Gene Bank Accession No. H36826).

In an additional investigation the gene Flowering Promoting Factor1 (FPF1) was characterized (Kania et al. supra) which is expressed in the apical meristem immediately after the photoperiodic induction of the flowers in the long-day plants Sinapis alba and Arabidopsis thaliana. In earlier transitional stages expression of FPF1 is only demonstrable in the peripheral zone of the apical meristem. Later however it can also be demonstrated in the flowering meristem and axillary meristem which form secondary inflorescences. The FPF1 gene codes a 12.6 kDa size protein which has no homologies to any previously identified protein with known function. A constitutive expression of the gene in Arabidopsis under the control of the CaMV 35S promoter resulted in a dominantly inheritable property of early flowering under short-day as well as long-day conditions. Treatments with gibberlin (GA) and paclobutrazol, an inhibitor of GA synthesis, have shown that FPF1 is involved in a GA-dependent signal path and modulates a GA response in apical meristems during the transition to flowering.

The three genes MADSA, MADSB, and FPF1 already characterized lead, in the case of constitutive expression, to an advanced flowering in Arabidopsis. The transgenic plants which overexpress MADSA or FPF1 show therein a completely normal yield of flowers and seed. In the case of 35S::AtMADSB lines in which the transgene is strongly expressed there is occasionally also a disposition of fused terminal flowers.

It is the objective of the present invention to regulate the time of flowering in useful and ornamental plants taking into account a graduated development of the plants.

This objective is realized according to the invention by overexpression of the combined genes MADSA and FPF1 or MADSB and FPF1 which are activated by the cauliflower mosaic virus (CaMV) 35S promoter in the entire plant including the apical meristem and thus induce a premature flowering without affecting the yield and propensity to growth of the plants at the same time.

By constitutive expression of the three genes MADSA, MADSB, and FPFb 1 and their combination, regulation of flowering with maintenance of productivity is possible. The combination of the genes can be created by means of vectors which have several genes under the control of different promoters or by fusion proteins in which the effective domains of the individual proteins are under the control of a single promoter.

Since all three ectopically expressed genes (MADSA, MADSB, and FPF1) lead to an advanced flowering, it was first of all investigated whether the effects of the three genes are expressed in plants which express two of the genes constitutively. For initial investigations crosses between plant lines were carried out which express the various genes constitutively. Along with MADSA, MADSB, and FPF1 the flowering meristem identity genes LFY and AP1 were also included in the investigations for this purpose.

Transgenic 35S::LFY plants develop, in contradistinction to wild-type plants, flowers even in the axes of the rosette leaves. The number of rosette leaves on the contrary is not reduced. The disposition of flowers on the apical meristem of Arabidopsis is coupled in wild-type plants with an internodal elongation (so-called bolting) of the main axis (Hempel and Feldman, Planta 192, 276-286, 1994). In 35S::LFY plants we find flowering without a previous extension of the main axis. Since 35S::FPF1 plants show a premature extension of the main axis before flowering, it is obvious that a constitutive expression of LFY does not lead to an activation of FPF1. After crossing transgenic 35S::LFY plants with transgenic 35S::FPF1 plants the offspring, which overexpress both genes constitutively, show once again a coordinated flowering and bolting. The number of rosette leaves in the 35S::LFY and 35S::FPF1 plants is in this case clearly reduced in comparison to 35S::FPF1 plants under long-day as well as short-day conditions.

It has been shown that the constitutive expression of AP1 leads to flowering dependent on the photoperiod (Mandel and Yanofsky, supra). The weak expression of AP1 leads on the contrary to a reduction of the vegetative phase under long-day conditions but has hardly any effects under short-day conditions, that is, the plants flower in short days only insignificantly earlier than wild-type plants. If 35S::FPF1 is crossed into a weakly expressing 35S::AP1 line then the offspring which express both genes constitutively flower under short-day conditions just as quickly as under long-day conditions. The influence of the photoperiod on flowering was thus increased once again. The observed changes of the time of flowering are in this case not additive but rather synergistic effects are observed. This can be explained by an increased competency of the transgenic 35S::FPF1 plants for action of the flowering meristem identity gene AP1.

Also after crossing of transgenic 35S::FPF1 plants with 35S::MADSA and 35S::MADSB plants it was shown that the offspring flower still earlier if FPF1 and one of the other genes are overexpressed at the same time. If MADSA and MADSB are overexpressed at the same time, then the offspring flower but not earlier than their respective parent plants. This indicates that these two genes are active in the same signal transduction path.

It could be shown that plants which express FPF1 constitutively become more competent for flowering, that is, they react more sensitively to the additional expression of the flowering meristem identity gene LFY and AP1 as well as to the expression of MADSA and MADSB. Since the three genes FPF1, MADSA, and MADSB in the case of an overexpression with a moderate amount of transcript do not restrict the fertility of the plants or their vitality and the observed influence on the flowering is additive, the prerequisites are provided hereby which make possible the regulation of the time of flowering in useful and ornamental plants to an extent previously unknown. As examples of such useful plants are, among others, plants of the genera Triticum, Oryza, Zea, Hordeum, Sorghum, Avena, Secale, Lolium, Festuca, Lotus, Medicago, Glycine, Brassica, Solanum, Beta, as well as plants producing vegetables or fruits and angiospermic trees are to be mentioned.

For the combination of the genes various possibilities present themselves. The genes can be combined into one transformation vector and regulated by different promoters. The use of different promoters is important since it was observed that genes which are regulated by identical promoters in transgenic plants can be partially suppressed by mechanisms which one summarizes under the overall concept of “cosuppression” (Matzke et al., Plant Journal 9, 183-194, 1996). The genes can also stand as fusion proteins in common under the control of a single promoter. The different possible combinations are presented in the examples below.

It is an additional objective of the present invention to make available transgenic plants which express MADSA and MADSB in antisense orientation. It could be shown that 35S::ASMADSA and 35S::ASMADSB clearly flower later than corresponding control plants. After crossing MADSA and MADSB antisense lines a still later flowering was observed in plants which express both antisense constructs. The delay of flowering correlates in this case directly to the strength of the expression of the antisense constructs. Since the overexpression of FPF1 increases the competency of plants for flowering, the suppression of FPF1 expression conversely also leads to a reduction of the competency for flowering. Thus transgenic lines could be selected which express FPF1 in antisense orientation and thereby clearly flower later than corresponding control plants. A selection of suitable lines which express different antisense constructs thus make possible a complete suppression of flowering.

In the case of plants from which as a rule only the vegetative parts are harvested, antisense constructs can be used in order to prevent an undesired flowering. This plays a great role in the case of sugar beets which store sugar in the beets only in the vegetative state. At the onset of flowering this sugar is once again mobilized and used for the development of inflorescence. Thereby not insignificant losses in the harvest result. Since hybrid seed stock is sown for the cultivation of sugar beets it must be ensured that the parent plants still flower in order to produce the seed stock. Here a strategy presents itself in which both parent parts are transformed with different constructs which lead in themselves alone to no noteworthy reduction of flowering. Then only the cooperation of both constructs in the hybrid plants leads to the suppression of undesired flowering which would lead to losses in yield. Inducible promoters or promoters which only become active through an activator from one of the parent plants, as have been described by Moore, I. et al., Proc. Natl. Acad. Sci. USA 95, 376-381, 1998 offer an additional possibility for permitting the expression only in the hybrid plants.

The present invention will be illustrated by the following examples.

### EXAMPLE I

### Production of Transgenic Arabidopsis Lines

1.1 Cloning of the Genes

The cloning of MADSA and MADSB was done as described in Menzel et al., Plant Journal 9, 399-408, 1996. FPF1 was cloned according to the process described in Kania et al., Plant Cell 9, 1327-1338.

1.2 Overexpression of the Genes FPF1:

The overexpression of FPF1 was performed according to the process described in WO 97/25433. Sense constructs of MADSA and MADSB:

For the overexpression of MADSA and MADSB cDNAs from mustard and Arabidopsis the coding regions of the cDNAs were amplified by the PCR process according to Ausubel et al., Current Protocols in Molecular Biology, Green Publishing Associates Wiley Interscience, New York, (1989). For the PCR the following primers were used:

MADSA:

(SEQ ID NO. 1)

| | |
|---|---|
| SaAEN: | 5′-CCGAATTCCATGGTGAGGGGAAAAACA-3′ |

(SEQ ID NO. 2)

| | |
|---|---|
| AtAEN: | 5′-CCGAATTCCATGGTGAGGGGCAAAACT-3′ |

(SEQ ID NO. 3)

| | |
|---|---|
| SaAEB: | 5′-CCGAATTCGGATCCTCACTTTCTGGAAGAACA-3′ |

(SEQ ID NO. 4)

| | |
|---|---|
| AtAEB: | 5′-CCGAATTCGGATCCTCACTTTCTTGAAGAACA-3′ |
| MADSB: | |

(SEQ ID NO. 5)

| | |
|---|---|
| SaBEN: | 5′-CCGAATTCCATGGGAAGGGGTAGGGTT-3′ |

(SEQ ID NO. 6)

| | |
|---|---|
| AtBEN: | 5′-CCGAATTCCATGGGAAGAGGTAGGGTT-3′ |

(SEQ ID NO. 7)

| | |
|---|---|
| SaBEB: | 5′-CCGAATTCGGATCCCTACTCGTTCGAAGTGGT-3′ |

(SEQ ID NO. 8)

| | |
|---|---|
| AtBEB: | 5′-CCGAATTCGGATCCCTACTCGTTCGTAGTGGT-3′ |

Along with the homologous region each of the primers AEN and BEN also contains an EcoRI and NcoI cut point and the primers AEB and BEB contain an EcoRI and a BamHI cut point. After EcoRI digestion the amplified products were ligated into the EcoRI cut point of the vector pBS SK+ (Stratagene). The insertions of selected clones were sequenced in order to rule out possible errors of the PCR. The coding regions were subsequently cut from the vector with NcoI and BamHi, purified over an agarose gel, and inserted into the vector pSH9 (Holtorf et al., Plant Mol. Biol., 29, 637-646, 1995). This vector contains the 35S promoter and the polyadenyl ligation signal from the cauliflower mosaic virus These so-called expression cassettes were subsequently cut with HindIII and ligated into the binary vector BIN19 (Bevan, Nucl. Acids Res. 12, 8711-8721, 1984). After multiplication of the recombinant plasmids in E. coli they were transformed into agrobacteria (Höfgen, R. and Willmitzer, L., Nucl. Acids Res. 16, 9877, 1988). Agrobacteria that contained the recombinant plasmids were used for the plant transformation.

1.3 Transformation of Arabidopsis.

The transformation of Arabidopsis was done by vacuum infiltration analogously to the process described according to Bechthold et al., C. R. Acad. Sci., Paris, 316, 1194-1199, 1993.

1.4 Evaluation of Transgenic Lines

Ten transgenic lines were investigated from each overexpression construct (sense). Since the length of the vegetative phase in the case of Arabidopsis correlates to the number of the rosette leaves formed, the number of leaves formed until flowering is evaluated as a measure of the time of flowering as a rule.

| Genotype | Leaves in Short Days | Leaves in Long Days |
|---|---|---|
| Col WT | 65.1 | 16.3 |
| 35S::SaMADSA7 | 36.7 | 12.3 |
| 35S::SaMADSB21 | 37.4 | 7.0 |
| 35S::AtMADSA1 | 11.2 | 7.5 |
| 35S::AtMADSB1 | 10.3 | 7.2 |
| 35S::AtFPF1 | 43.3 | 11.4 |
| 35S::SaLFY | 46.6 | 14.2 |
| 35S::SaAP1 | 58.4 | 9.6 |

From the 10 transgenic lines evaluated per construct the values of the earliest flowering line were listed in the table. The total number of leaves for each is entered, including the high leaves on the main axis of inflorescence.

From the table it can be seen that the transgenic plants in both photoperiods clearly produce fewer leaves and thus also flower earlier than the control plants. The particularly early flowering of the lines which overexpress the Arabidopsis cDNAs of MADSA and MADSB is striking. This resulted due to a better transformation yield so that in contradistinction to transformation with SaMADSA and SaMADSB cDNAs can even be selected after early flowering under the primary transformants.

### EXAMPLE II

### Crossing of Lines which Overexpress Different Genes

2.1 Crossing Experiments

Crossing of the following lines was carried out.

35S::AtFPF1 X 35S::SaMADSA

35S::AtFPF1 X 35S::SaMADSB

35S::SaMADSA X 35S::SaMADSB

35S::AtFPF1 X 35S::SaAPI

35S::AtFPF1 X 35S::SaLFY

For the crossings the still closed flower buds of the recipient plants were opened with pincers and the pollen sack of the flowers was removed in order to avoid self-pollination. The pollen of the lines cited above as the first was then transferred to each of the pods of the opened buds. After 4 weeks ripe were harvested and the seeds sown for the additional investigations.

2.2 Evaluation of the Crossings

| | Leaves in | Leaves in |
|---|---|---|
| Genotype | Short Days | Long Days |
| Col WT | 65.1 | 16.3 |
| 35S::AtFPF1 × 35S::SaMADSA | 16.2 | 6.3 |
| 35S::AtFPF1 × 35S::SaMADSB | 11.5 | 7.8 |
| 35S:: SaMADSA × 35S::SaMADSB | 35.5 | 10.1 |
| 35S::AtFPF1 × 35S::SaAPI | 9.8 | 8.6 |
| 35S::AtFPF1 × 35S::SaLFY | 17.3 | 11.6 |

From the crossing of the transgenic plants double homozygotous lines were initially selected. Twelve plants were drawn and evaluated from each of the selected lines.

In all the lines into which 35S::AtFPF1 had been crossed a clear reduction of the time period until flowering was shown. The plants which overexpressed the MADSA and MADSB showed no additional shortening of the vegetative phase.

### EXAMPLE III

### Production of Plant Lines with two Transgenes

3.1 Production of Transformation Vectors which Contain two Genes under the Control of Different Promoters

The coding regions of SaMADSA, SaMADSB, and AtFPF1 were ligated into the pSH5 vector which contains a ubiquitin promoter (Holtorf et al., supra), as described as in Example I. The cloned expression cassettes were then cut with PstI, purified over a gel, and ligated into the pBS SK^{[illegible]} vector. The fragments could be cut from the pBS SK^{[illegible]} vector with the ubiquitin promoter, then the respective coding region and the CaMV terminator with BamHI and EcoRI, and inserted into the corresponding pBIN19 MADSA , pBIN19 MADSB , and pBIN19 FPF1 vectors in which the coding regions of the corresponding genes are controlled by the CaMV promoter. The transformation vectors pBIN19 MADSA MADSB, pBIN19 MADSA FPF1, and pBIN19 MADSB FPF1 were obtained. These vectors were subsequently multiplied in E. coli and transformed into agrobacteria. Arabidopsis plants were transformed with the infiltration method according to Bechthold et al. (supra).

3.2 Analysis of the Transgenic Plants

| | Leaves | Leaves in |
|---|---|---|
| Genotype | in Short Days | Long Days |
| Col WT | 65.1 | 16.3 |
| 35S::AtMADSA-UBI::AtMADSB | 38.3 | 12.1 |
| 35S::AtMADSA-UBI::AtFPF1 | 18.2 | 8.4 |
| 35S:: AtMADSB-UBI::AtFPF1 | 19.6 | 7.8 |

Also in this experiment it has been shown that plants with two transgenes clearly flower earlier than the control plants. A selection of various times of flowering from a plurality of independent transformants was furthermore possible.

### EXAMPLE IV

### Production of Transformation Vectors with Fusion Proteins between FPF1 and MADSA or FPF1 and MADSB.

4.1 Production of the Constructs and Transformation of Plants

PCR fragments of the coding regions of MADSA, MADSB, and FPF1, each of which has an NcoI cut point at the start codon and after the last coded amino acid, are introduced into the recombinant vectors pBIN19 MADSA, pBIN19 MADSB, and pBIN19 FPF1 at the NcoI cut point. Thereby recombinant vectors were generated which contained two coding regions under the control of the CaMV promoter. The four constructs 35S::MADSA::FPF1, 35S::MADSB::FPF1, 35S::FPF1::MADSA, and 35S::FPF1::MADSB were obtained. The recombinant vectors were multiplied in E. coli and transferred into agrobacteria. Arabidopsis was transformed according to Bechthold et al. (supra).

4.2 Analysis of the Transgenic Plants

The transgenic plants clearly flower earlier than corresponding control plants and than plants which each overexpress only one gene. Ten plants from each of 8 transformed lines were evaluated. The values of the earliest lines are presented in the table.

| | Leaves in | Leaves in |
|---|---|---|
| Genotype | Short Days | Long Days |
| Col WT | 65.1 | 16.3 |
| 35S::AtMADSA::AtFPF1 | 21.3 | 11.2 |
| 35S::AtMADSB::AtFPF1 | 18.2 | 10.8 |
| 35S::AtFPF1::AtMADSA | 23.8 | 12.1 |
| 35S::AtFPF1::AtMADSB | 22.7 | 12.3 |

In this experiment it has been shown that plants with two transgenes under the control of only one promoter also clearly flower earlier than the control plants. A selection of various times of flowering from a plurality of independent transformants was likewise also possible.

### EXAMPLE V

### Change of the Time of Flowering in Transgenic Tobacco Varieties with Different Photoperiodic Dependencies for the Induction of Flowering

Since the discovery of the photoperiodic induction of flowering (Garner and Allard, J. Agric. Res. 18, 553-606, 1920) countless studies have been carried out in order to understand the influence of the length of the day on the induction of flowering. Most of the types of plants which were used for this purpose show a strict dependence on the photoperiod, that is, they only flower if a critical duration of the light period is exceeded (long-day plants) or not exceeded (short-day plants). Among these plants are in particular also the different types of tobacco with different photoperiodic requirements for an induction of flowering. In the examples described here three different types of tobacco were used, the long-day tobacco Nicotiana sylvestris (Ns), the day-neutral tobacco Nicotiana tabacum (Nt), and the short-day tobacco Nicotiana tabacum Maryland Mammoth (Nt-MM). Through the use of the gene construct presented in the preceding examples an induction of flowering for the strictly photoperiodic tobacco varieties can also be accomplished under non-inducing photoperiods.

5.1 Transformation of Tobacco

For the transformation of the various photoperiodic tobacco varieties the constructs were used which were described in Example I. In addition the homologous FPF1 gene from Nicotiana tabacum was still used which has a identity of the nucleotide sequence in the coding region of 67.7% to the FPF1 gene from mustard. This tobacco FPF gene was provided in the same manner for a constitutive expression with a CaMV promoter and a terminator as was described in Example I for mustard and Arabidopsis transgenes. For this purpose an Nco1 restriction cut point at the start codon and a BamHI restriction cut point at the stop codon was introduced by a PCR reaction with the following primers.

(SEQ ID NO. 9)

| | |
|---|---|
| NtFPF-EN: | 5′ CAGGAATTCCATGGCTGGAGTTTGGGT 3′ |

(SEQ ID NO. 10)

| | |
|---|---|
| NtFPF-EB: | 5′ CAGGAATTCGGATCCTTATCATATGTCTCTAAC 3′ |

The transformation of tobacco was carried out with a standard method (Horsch et al., Science 227, 1229-1234, 1985).

5.2 Constitutive Expression of FPF1, MADSA, or MADSB

The transgenic plants were under the same short-day or long-day conditions in a controlled cabinet as were used for Arabidopsis.

For the evaluation of the time of flowering the period of time from sowing until the opening of the first flower was used in the case of the tobacco. From each of the represented lines 8 plants were evaluated. In the following table plants are considered which each overexpress only one gene constitutively.

| | Number of | |
|---|---|---|
| | Days Until | Number of Days |
| | Flowering | Until Flowering |
| Genotype | in Short Days | in Long Days |
| Nt | 93 | 76 |
| Nt 35S::SaFPF1 | 85 | 68 |
| Nt 35S::SaMADSA | 76 | 55 |
| Nt 35S::SaMADSB | 68 | 54 |
| Nt 35S::NtFPF1 | 81 | 64 |
| Nt-MM | 106 | non-flowering |
| Nt-MM 35S::S FPF1 | 99 | non-flowering |
| Nt-MM 35S::SaMADSA | 72 | 124 |
| Nt-MM 35S::SaMADSB | 80 | non-flowering |
| Nt-MM 35S: NtFPF1 | 97 | non-flowering |
| Ns | non-flowering | 82 |
| Ns 35S::FPF1 | non-flowering | 78 |
| Ns 35S::MADSA | non-flowering | 76 |
| Ns 35S::MADSB | 94 | 70 |
| Ns 35S::NtFPF1 | non-flowering | 67 |

The evaluation of this experiment shows that the day-neutral tobacco Nicotiana tabacum comes to flower through the overexpression of the various transgenes under short-day conditions as well as long-day conditions. The flowering and seed yield is in all cases comparable to the yield in the wild-type plants. The transgenic short-day tobacco Nicotiana tabacum Maryland Mammoth flowers under inducing short-day conditions each time earlier than the wild-type plants under the same conditions. Under non-inducing long-day conditions the wild-type Maryland Mammoth tobacco does not flower. Transgenic Maryland Mammoth tobacco which overexpresses FPF1 or MADSB also does not flower under long-day conditions, but if MADSA is overexpressed, then this tobacco also flowers under non-inducing conditions. By overexpression of only a single gene the photoperiodic confines of the induction of flowering under non-inducing conditions has been overcome. Nicotiana sylvestris wild-type plants do not flower under short-day conditions and also the constitutive expression of FPF1 or MADSA does not lead to flowering under non-inducing conditions. The overexpression of MADSB however does also lead to flowering under non-inducing short-day conditions in the long-day tobacco Nicotiana sylvestris.

### EXAMPLE VI

6.1 Combined Expression of FPF1 with MADSA or MADSB in the Different Varieties of Tobacco

Analogously to the combinations of transgenes by crossings described in Example II, crossings were also carried out with the various photoperiodic tobacco lines which overexpress FPF1, MADSA, or MADSB. In the following table the times of flowering of plants which each contain two transgenes are listed.

| | Number of | Number of |
|---|---|---|
| | Days Until | Days Until |
| | Flowering | Flowering |
| Genotype | in Short Days | in Long Days |
| Nt 35S::SaFPF1 × Nt 35S::SaMADSA | 65 | 59 |
| Nt 35S::FPF1 × Nt 35S::SaMADSB | 60 | 50 |
| Nt-MM 35S::SaFPF1 × Nt-MM | 68 | 88 |
| 35S::SaMADSA | | |
| Nt-MM 35S::SaFPF1 × Nt-MM | 76 | 98 |
| 35S::SaMADSB | | |
| Nt 35S::SaFPF1 × Nt 35S::SaMADSA | non-flowering | 67 |
| Nt 35S::SaFPF1 × Nt 35S::SaMADSB | 82 | 62 |

Until up to the crossing of Ns-SaFPF1 with Ns-SaMADSA the combined expression leads in all cases to the vegetative phases being shortened further. While Maryland Mammoth plants which overexpress either MADSB or FPF1 do not initiate flowering under non-inducing conditions, the combined expression of these two genes under otherwise equal conditions leads to flowering.

### EXAMPLE VII

### Modification of the Time of Flowering in Rape Plants

Rape is an agronomically important plant which is cultivated on all continents for the production of culinary and industrial oils. In the northern latitudes, such as e.g., in Canada or Scandinavia, there is in rape-cultivating regions the danger of early onset of winter which frequently degrades the rape harvest since the rape cannot then mature and only provides low-quality oil. An advance of the time of flowering and thus an earlier maturity of the rape plants by a few days could solve this problem. Furthermore, early-blooming rape plants can be cultivated still further north and thus the arable area extended.

7.1 Production of Transgenic Rape Plants

For an overexpression in rape plants (Brassica napus) the vectors for the expression of FPF1, MADSA, and MADSB described in Example I are used. The transformation was accomplished according to a standard method (Moloney, et al., Plant Cell Reports, 8, 238-242, 1989). A winter (WR) and a summer (SR) rape line were transformed.

7.2 Analysis of the Time of Flowering of the Transgenic Rape Plants

The number of days which the rape was ripe earlier than corresponding control plants was recorded in the table. Twelve plants were evaluated from each represented transgenic line. The plants were cultivated in greenhouses and as is necessary in the case of winter rape exposed to vernalization conditions for different times.

| | Number of Days by which the |
|---|---|
| Genotype | Transgenic Rape Ripened Earlier |
| Bn (WR) 35S::SaFPF1 | 7 |
| Bn (SR) 35S::SaFPF1 | 3 |
| Bn (WR) 35S::SaMADSA | 7 |
| Bn (SR) 35S::SaMADSA | 5 |
| Bn (WR) 35S::SaMADSB | 12 |
| Bn (SR) 35S::SaMADSB | 6 |

The transgenic rape plants were mature significantly earlier than the wild-type plants under the same conditions. It has furthermore been shown that winter rape plants which overexpress the MADSB gene clearly have to be vernalized more briefly in order to arrive at flowering. While wild-type plants have to be held at 4° C. for 8 weeks, only 2 weeks vernalization was necessary for 35S::MADSB plants for complete competency for flowering. The combination of 35S:MADSB with 35S::FPF1 led in this case even to a complete elimination of the vernalization requirement for flowering. This can be utilized for the rapid cultivation of winter grains and winter rape plants or for sowing of the seeds after the winter period.

### EXAMPLE VIII

### Production of Transformation Vectors with Antisense Constructs for the Prevention of Flowering

8.1 Production of Transformation Vectors with Antisense Constructs of FPF1, MADSA, and MADSB

The antisense constructs find application, for example, in the cultivation of sugar beets and salad plants. The process here was carried out modeled on Arabidopsis thaliana.

Antisense Constructs:

Through the transformation of plants with DNA constructs which make possible the transcription of an antisense RNA in the plant, the expression of a gene can be suppressed so that from the phenotypic changes of the transformed plant which may occur the function of this gene in processes of material exchange or development can be deduced. In order to achieve a specific inhibition of the expression of AthMADSA and AthMADSB without a simultaneous influence of the activity of other MADS Box genes, those sections of the Arabidopsis cDNAs were used for the production of the transformation constructs which did not contain the conserved MADS Box region. In a first step a 530 bp-long XbaI/HindIII fragment of the AthMADSA cDNA which contains a portion of the coding region and the almost complete 3′ non-coding region as well as a 640 bp-long BamHI/HindIII fragment of the AthMADSB cDNA which also contains a portion of the coding region and the complete 3′ non-coding section was cut. The projecting ends of the isolated fragments were filled out and ligated into the Sma I cut point of the pBS SK+ vector (Stratagene).

For the production of the FPF1 antisense construct the complete cDNA with BamHI and EcoRI could be cut from out of a pBS SK+ vector in the correct orientation.

According to the determination of suitable orientation of the MADSA and MADSB cDNAs all three cDNAs could be isolated with BamHI and EcoRI and, directed in antisense orientation, ligated into the vector pRT104 (Töpfer et al., Nucl. Acids Res. 15, 5890, 1987). Thereby a promoter::antisense::terminator cassette arose consisting of the CaMV 35S promoter, the respective cDNA (MADSA, MADSB, or FPF1), and a CaMV polyadenyl ligation signal. For checking of the antisense orientation of the cDNA fragments the constructs were sequenced.

The antisense constructs were then isolated by HindIII digestion from the vector prt104 and inserted into the HindIII cut point of the plant transformation vector pBIN19 (Bevan, supra). The individual steps of the cloning were pursued by southern blot analyses.

The recombinant BIN19 plasmids were cloned in E. coli and subsequently transferred into agrobacteria. Arabidopsis was transformed according to Bechthold et al., (supra).

8.2 Analysis of the Transgenic Plants

| Genotype | Leaves in Short Days | Leaves in Long Days |
|---|---|---|
| Col WT | 65.1 | 16.3 |
| 35S::ASAtFPF1 | 79.8 | 18.2 |
| 35S:: ASAtMADSA40 | 73.3 | 21.0 |
| 35S::ASAtMADSB74 | 76.5 | 17.9 |
| 35S:: ASAtMADSA40 × | 86.3 | 25.8 |
| 35S::ASAtMADSB74 | | |

It could thus be shown that transgenic lines with antisense constructs clearly flower later than corresponding control plants.

1. A method of delaying induction of flowering in a plant, the method comprising expressing two sequences of different flowering induction genes in said plant, wherein said sequences are operably linked to either one regulatory sequence, or independently to separate regulatory sequences, in each case in antisense orientation, and wherein said regulatory sequence(s) drive expression in said plant of said sequences of flowering induction genes are operably linked thereto.

2. The method according to claim 1, wherein the plant expressing two sequences of different flowering induction genes is obtained by crossing two parental plants, wherein each parental plant expresses a sequences of a different flowering induction gene, and wherein the plant expressing two sequences of different flowering induction genes is selected among progeny of said parental plants.

3. The method according to claim 2, wherein the expression of the sequences of the flowering induction genes in the parental plants does not cause a noteworthy reduction of flowering in said parental plants.

4. The method according to claim 2, wherein the sequences of flowering induction genes, which are operably linked in antisense orientation to a regulatory sequence, are introduced into said parental plants by infection with Agrobacteria.

5. The method according to claim 1, wherein said method comprises the steps of:
(a) introducing a plant a recombinant DNA molecule, wherein said recombinant DNA molecule comprises a regulatory sequence and two flowering induction gene sequences, wherein said flowering induction gene sequences are operably linked to the regulatory sequence in antisense orientation, and wherein said regulatory sequence drives expression in said plant of the sequences operably linked thereto, and
(b) regenerating said plant, wherein antisense RNA is expressed in the resulting regenerated plant so as to delay the induction of flowering in said plant.

6. The method according to claim 1, wherein said two sequences of different flowering induction genes are selected from the group consisting of SEQ ID NO. 11, SEQ ID NO.12, SEQ ID NO. 13, SEQ ID NO. 14, SEQ ID NO. 15, SEQ ID NO. 16, SEQ ID NO. 17, and SEQ ID NO. 18 and wherein neither of said two sequences contain a nucleic acid sequence encoding a MADS Box domain.

7. The method according to claim 1 wherein said sequences of two flowering induction genes sequences comprises a Xbal/HindIII restriction fragment of a MADSA gene from Arabidopsis thaliana.

8. The method according to claim 7, wherein said restriction fragment is approximately 534 base pairs in length.

9. The method according to claim 8, wherein said restriction fragment is depicted in SEQ ID NOS. 13 and 17.

10. The method according to claim 1, wherein one of said two flowering induction gene sequences comprises a HindIII/Xbal restriction fragment of a MADSB gene from Arabidopsis thaliana.

11. The method according to claim 10, wherein said restriction fragment is approximately 534 base pairs in length.

12. The method according to claim 11, wherein said restriction fragment is depicted in SEQ ID NO.

13. The method according to claim 5, wherein said recombinant DNA molecule comprises pBIN9 having inserted therein said regulatory sequence and said at least two flowering induction gene sequences, wherein said at a least two flowering induction gene sequences are operably linked to the regulatory sequence in antisense orientation.

14. The method according to claim 5, wherein said recombinant DNA molecule is introduced into said plant by infection with Agrobacteria.

15. The method according to claim 1, wherein said plant is an ornamental plant.

16. The method according to claim 1, wherein said plant is a crop plant.

17. The method according to claim 1, wherein said plant is a sugar beet plant. 1.
